(19) United States
(12) Patent Application Publication
(10) Pub. No.: US 2021/0121389 A1
(43) Pub. Date: Apr. 29, 2021

(54) PRODUCT FOR COSMETIC SKIN CARE

(71) Applicant: S.I.P.R.E.S. S.R.L., Limena (IT)

(72) Inventors: Antonio CATALANO, Padova (IT); Khalil MITRI, Padova (IT)

(21) Appl. No.: 17/068,286

(22) Filed: Oct. 12, 2020

(30) Foreign Application Priority Data
Oct. 25, 2019 (IT) 102019000019772

(51) Int. Cl. A61K 8/81; A61K 8/04; A61K 8/19; A61Q 19/00

(52) U.S. Cl. CPC A61K 8/8152; A61K 8/042; A61K 2800/87; A61Q 19/001; A61K 2800/47; A61K 8/19

(57) ABSTRACT

A product for cosmetic skin care includes, comprising an applicator and an active-care cosmetic composition.

The applicator is constituted by an ergonomic metallic body adapted for skin contact, in which a permanent magnet with magnetic field strength substantially comprised of an amount between 0.1 mT and 10 mT is at least partially embedded.

### CROSS-REFERENCE TO RELATED APPLICATIONS

This application is related to and claims the benefit of Italian Patent Application No. 102019000019772, filed on Oct. 25, 2019, the content of which are herein incorporated by reference in their entirety.

### TECHNICAL FIELD

The present disclosure relates to a product for cosmetic skin care, comprising an applicator and an active-care cosmetic composition.

### BACKGROUND

The diamagnetic properties of some molecules are known and used to increase their skin penetration.

However, some particularly beneficial molecules have weak diamagnetic properties (diamagnetic susceptibility between −0.1×10⁻⁸ m³/kg and −350×10⁻⁸ m³/kg) and therefore it would be necessary to use very intense magnetic fields in order to take advantage of them, with expenditure of energy, low practicality and possible side effects.

WO2016/044552A1 describes:

an applicator comprising a unidirectional magnetic array with a pitch comprised between 1.7 mm and 2.5 mm and a magnetic field strength between 24.0 and 36.0 mT and

a skin care composition comprising the peptide palmitoyl-lysine-threonine-threonine-lysine-serine (Pal-KTTKS) and a dermatologically acceptable carrier.

In WO2016/044552A1 the skin care composition described can improve the appearance of visible and/or tactile blemishes in human skin, including fine lines, wrinkles, dilated pores, roughness, dryness and other blemishes in the skin structure, for example, to reduce or erase the visibility of thin lines, wrinkles and other forms of irregular or rough surface texture associated with aged or photodamaged skin.

The skin care composition can take various different forms such as, for example, solutions, suspensions, lotions, creams, gels, toners, sticks, pencils, sprays, aerosols, ointments, cleansing liquid washes and solid bars, shampoos and hair conditioners, pastes, foams, powders, mousses, shaving creams, wipes, strips, patches, electrically-powdered patches, wound dressing and adhesive bandages, hydrogels, film-forming products, facial and skin masks, cosmetics like foundations, eyeliners, eye shadows and the like.

For example, the cosmetic composition can be applied for the skin care of the face that requires treatment.

The surface of the skin of the face can include one or more areas of the cheek, the forehead and the eye contour areas.

One or more of these skin surfaces can be identified as in need of treatment when signs of skin aging are observed on the target skin surface.

The applicator of WO2016/044552A1 improves the skin penetration of the composition by taking advantage of its diamagnetic characteristics and therefore the repulsive force generated on it by the magnetic field of the applicator.

However, some particularly beneficial molecules, such as, for example, collagen hydrolysates, amino acids, DNA and DNA hydrolysates, metals like silver, gold, copper and organic substances associated with such metals, and plant oils, have weak diamagnetic properties, and therefore it would be necessary to use very intense magnetic fields in order to take advantage of them, with expenditure of energy, low practicality and possible side effects.

### SUMMARY

The aim of the present disclosure is to provide a skin care product, comprising an applicator and an active-care cosmetic composition, which is capable of improving the known art in one or more of the above mentioned aspects.

Within this aim, the disclosure provides a skin care product with which it is possible to deliver weakly diamagnetic active substances without using excessively strong magnetic fields.

The disclosure further provides a skin care product that enables a greater penetration of the active ingredient.

The present disclosure overcomes the drawbacks of the known art in an alternative manner to any existing solutions.

The disclosure provides a skin care product that is highly reliable, easy to implement and of low cost.

This aim and these and other advantages which will become better apparent hereinafter are achieved by providing a product for cosmetic skin care, comprising:

an applicator, constituted by an ergonomic metallic body adapted for skin contact, in which a permanent magnet with magnetic field strength substantially comprised between 0.1 mT and 10 mT is at least partially embedded, and

a cosmetic composition comprising a mesh of polymers which incorporates water.

### BRIEF DESCRIPTION OF THE DRAWINGS

Further characteristics and advantages of the disclosure will become better apparent from the description of a preferred, but not exclusive, embodiment, which is illustrated by way of non-limiting example in the accompanying drawings wherein:

FIGS. 1-3 are different views of an applicator comprised in the product according to the disclosure; and

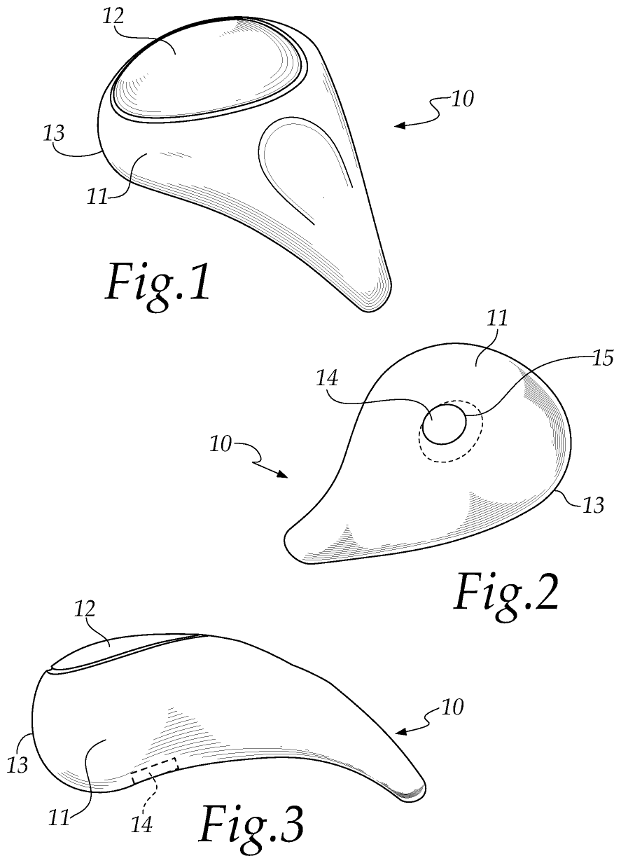

FIGS. 4 and 5 show the method of application of the product.

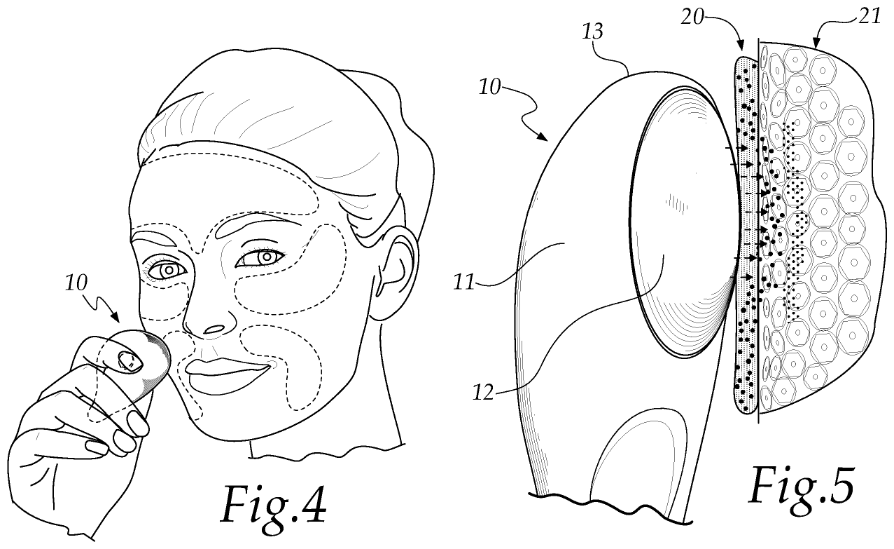

### DETAILED DESCRIPTION OF THE DRAWINGS

With reference to the figures, a product for cosmetic skin care, according to the disclosure, comprises an applicator 10 which is constituted by a metallic body 11, which is ergonomic and adapted to be handled, as well as adapted for skin contact, for example substantially teardrop-shaped, like the one illustrated in the figures.

Because of this, the surface of the body 10 has a wide region 12 with a broad curvature adapted for contact with the face and neck and a thinner region 13 for the outline of the eyes, lips and nose.

The body 11 is conveniently made of AISI 316L steel, therefore with a low carbon content and, as a consequence, is adapted for contact with human skin because it is particularly resistant to corrosion.

Obviously, alternatively other metals compatible with application on the skin can be used.

A permanent magnet 14 with magnetic field strength substantially comprised between 0.1 mT and 10 mT is partially embedded in the body 11.

In the example in the figures, the permanent magnet 14 is disc-shaped and is accommodated in a hollow, complementarily shaped seat 15 of the body 11 which is opposite to the region 12 with broad curvature.

Advantageously, a neodymium permanent magnet is preferred, but other types of magnets can also be used which can maintain their magnetic charge over time.

In a variation of embodiment, not shown in the figures, the body 11 is hollow and the permanent magnet 14 is, at least partially, inserted inside it.

In this case, the product 10 is lighter.

Optionally, the zone 12 can have porosities, for example with diameter and depth between 0.05 mm and 2 mm.

The cosmetic composition 20 comprises a mesh of polymers which incorporates water.

Such cosmetic composition 20 is, for example, a substance in gel form that comprises:

between 0.1% and 5% by weight of sodium acrylate copolymer,

50% to 95% by weight of water.

As mentioned previously, the composition 20 is based on a mesh of polymers that incorporates water. Once spread on the skin 21, the mesh breaks, the water evaporates and this lowers the temperature of the skin 21.

The subsequent massage with the steel applicator 10 absorbs further heat from the skin, and this action is combined with the physical effect of diamagnetism.

As is known, diamagnetic materials are repulsed by a magnetic field.

On the basis of this principle, the applicator 10 uses the diamagnetic action to bring active ingredients deep into the skin 21 (see the arrows in FIG. 5).

By combining a composition that is capable of physically cooling the skin 21 during its application with the coupled magnetic applicator/weakly diamagnetic active ingredients, fundamental improvements are obtained in the skin care action 21.

In fact, the cooling brings about an immediate vasoconstriction of the blood circulatory system, by virtue of the activation of the afferent sensory nerves, of the lymphatic system and a reduction of the diameter of the pores of the skin 21.

Immediately afterward, however, there is a measurable rebound effect which, by contrast, activates the zone and thus enables a deeper penetration.

The physical massage caused by the use of the applicator accelerates the rebound effect.

The cooling leads to an activation of the afferent nerve fibers by way of activation of Transient Receptor Potential (TRP) ion and other non-mediated mechanisms.

The metallic component of the applicator 10 first contributes to absorb heat from the skin 21 by virtue of its thermal conductivity properties, and subsequently it heats up.

Overall weakly diamagnetic active ingredients can be conveyed without using excessively strong magnetic fields.

In practice it has been found that the disclosure fully achieves the intended aim and objects.

The disclosure thus conceived is susceptible of numerous modifications and variations, all of which are within the scope of the appended claims. Moreover, all the details may be substituted by other, technically equivalent elements.

In practice the materials employed, provided they are compatible with the specific use, and the contingent dimensions and shapes, may be any according to requirements and to the state of the art.

The cosmetic composition comprises a mesh of polymers which incorporates water.

1. A product for cosmetic skin care, comprising:
an applicator, constituted by an ergonomic metallic body adapted for skin contact, in which a permanent magnet with magnetic field strength substantially comprised between 0.1 mT and 10 mT is at least partially embedded, and
a cosmetic composition comprising a mesh of polymers which incorporates water.

2. The product according to claim 1, wherein said ergonomic metallic body is adapted to be handled.

3. The product according to claim 2, wherein said ergonomic metallic body is substantially teardrop-shaped and has a wide region with a broad curvature adapted for contact with face and neck and a thinner region for the outline of eyes, lips, and nose.

4. The product according to claim 1, wherein said ergonomic metallic body is made of corrosion-resistant steel and/or of other metals compatible with application on skin.

5. The product according to claim 3, wherein said permanent magnet is disc-shaped and is accommodated in a hollow, complementarily shaped seat of the ergonomic metallic body which is opposite to the wide region with broad curvature.

6. The product according to claim 1, wherein said permanent magnet is a neodymium magnet and/or is such as to maintain a magnetic charge over time.

7. The product according to claim 3, wherein said wide region with broad curvature of said ergonomic metallic body has a plurality of porosities.

8. The product according to claim 7, wherein said plurality of porosities have a diameter and depth of between 0.05 mm and 2 mm.

9. The product according to claim 1, wherein said cosmetic composition is a substance in gel form that comprises:
between 0.1% and 5% by weight of sodium acrylate copolymer, and
50% to 95% by weight of water.
